(19) United States
(12) Patent Application Publication
(10) Pub. No.: US 2015/0032063 A1
(43) Pub. Date: Jan. 29, 2015

(54) Components and devices for closed medical system operation

(71) Applicant: Gale Harrison Thorne, Bountiful, UT (US)

(72) Inventor: Gale Harrison Thorne, Bountiful, UT (US)

(21) Appl. No.: 14/121,681

(22) Filed: Oct. 7, 2014

Related U.S. Application Data
(63) Continuation-in-part of application No. 13/872,828, filed on Apr. 29, 2013.
Application No. 13/872,828 is a continuation-in-part of application No. 13/068,529, filed on May 13, 2011.

(51) Int. Cl. A61M 5/315; A61M 39/26

(52) U.S. Cl. CPC A61M 5/31596; A61M 39/26; A61M 2005/1787
USPC 604/207

(57) ABSTRACT

A self-closing slit valve which is opened by compression inside a receiving tapered female orifice. For luer fittings, internal taper of a female luer fitting provides sufficient distortion of an asymmetrical valve to distort a closed, preferably planar slit to a patent fluid pathway. The slit may be opened by valve displacement into a tapered female connector orifice or by being forced open by a blunt cannula. The valve may be effectively used as a valving part in needleless connector adapters. A special method for constructing the valve when imposed upon by restrictive tolerances of a luer fitting is also disclosed. Examples of uses of the self-closing valve in male luer needleless connector adapters and closed mixing systems and closed mixing systems with flush are also disclosed. An application providing a closed male luer adapter, a closed valved male luer replacement for a syringe is disclosed as well as applications associated with closed system mixing syringes.

### CONTINUITY

This application is a Continuation-in-Part of U.S. patent application Ser. No. 13/872,828, filed Apr. 29, 2013 and titled TWISTED SLIT VALVE filed by Gale H. Thorne (referenced hereafter as Thorne 828) and, further, a Continuation-in-Part of U.S. patent application Ser. No. 13/068,529 filed May 13, 2011 by Gale H. Thorne, et al. and titled PRESSURE ACTUATED VALVE FOR MULTI-CHAMBER SYRINGE APPLICATIONS (hereafter referenced as Thorne 529), contents of each of which are made part hereof, by reference.

### FIELD OF INVENTION

This application relates to tapered fitting systems which employ self-closing valves, in general, and to male valves opened by insertion into tapered female fittings, in particular, such valves being opened by compressive forces about the exterior of the valve and, once compressive force is removed, self close to stop fluid flow. Inventions disclosed within this application also relate, generally, to applications of such valves in male luer adapters for needleless interconnections, male luer replacement by such valves in otherwise conventional syringes and in novel closed system mixing syringes with and without integral flush.

### BACKGROUND AND DESCRIPTION OF RELATED ART

While the present invention broadly applies to self-closing valves which are opened by insertion into tapered fittings, it has particular application to self-sealing male and female luer valves used, for example, in the following medical applications.

### Example 1

### Male Adapter Valves

Two primary prior art patents well disclose the need and opportunity for a male luer valve which is opened upon insertion into a female luer fitting. The first, U.S. Pat. No. 7,766,304 B2 issued to John C. Phillips (Phillips 304) Aug. 3, 2010 and titled, SELF-SEALING MALE LUER CONNECTOR WITH BIASED VALVE PLUG discloses a male luer connector for connection with a female luer connector. Phillips 304 further discloses a device comprising a tubular male body and a surrounding displaceable cuff. A valve plug is slidably disposed within the housing and formed to, in a first state, seal a communicating hole and, in a second state, be displaced to open the hole for fluid communication. Closure is biased to occur by an elastomeric coupling which communicates with the plug.

The second, U.S. Pat. No. 7,803,140 B2 issued to Thomas F. Fangrow, et al (Fangrow 140) Aug. 16, 2011 and titled, MEDICAL CONNECTOR discloses two primary designs for a male luer connector for connection with a female luer connector. The first design comprises a plugging component which is offset to open a valve for fluid flow. The second design discloses a slit valve which is opened for flow by insertion of a piercing part.

Such male valves provide barriers for infecting bacteria and debris, but perhaps more importantly provide a self-closing barrier and, thus, a closed system against inadvertent leakage, wherein product associated with such leakage might be a hazardous drug. It is important to note that such male valves should only be disposed in an open state while the valve is inserted into a complementary female fitting. At this date, all contemporary commercial male adapters known to the inventor for needleless connectors employ either a linear displacement mechanism which removes a “plug” from a hole when the valve is inserted into a female luer fitting or a forceably opened slit. Such mechanisms are commonly complex in structure and, therefore, result in an elevated component cost. Generally within the scope and meaning of this Application, the term male luer adapter shall be used as a reference for such needleless connector devices.

Further, male luer adapters such as those provided as examples, supra, are actuated to an open state by either a displacement of a plug within a hole or by a slit of a valve being parted by insertion of a piercing part. In the case of plug displacement, such is known to often result in a small droplet of liquid remaining resident at the exterior of the hole and plug site upon closure.

### Example 2

### Luer Fitting Replacements on Otherwise Conventional Medical Syringes

The value of adding a male adapter fitting to a conventional medical syringe has been demonstrated by at least one company which currently sells one of the above cited male adapters by securely affixing a male adapter to a syringe and selling the combination as an integrated product. As is well understood in medical syringe art, definite advantages in cost and elimination of dead space would result by replacing a male luer fitting on a syringe with a male luer adapter.

### Example 3

### Closed System Mixing Syringes with Flush

U.S. Pat. No. 7,101,354 B2 issued Sep. 5, 2006 to Gale H. Thorne, Jr., et al., and titled MIXING SYRINGE WITH AND WITHOUT FLUSH (Thorne 354) discloses technology for making a mixing syringe with flush capability. However, Thorne 354 is silent regarding application to a closed system. Thorne 354 teaches and claims a removable cap which is used to restrain fluid within a syringe during a mixing process, but which must be removed prior to dispensing fluid from the syringe, terminating the opportunity for a closed system. Such a removable cap is detrimental to achieving closed system operation. Further, Thorne 354 teaches use of a one-way valve to separate a drug containing chamber from a diluent chamber. Such an approach must be considered unwise for, while the one-way valve permits pulsatile transfer of diluent from an intermediate chamber into a mixing chamber, such a one-way valve must be considered inadequate to keep drug and diluent disparate under all handling and transport conditions. It should be noted that any premature leakage of diluent into a drug containing mixing chamber within a mixing syringe could not be considered for a device for safe and efficient storage, transport and mixing of drugs. For these reasons, it is respectfully submitted that Thorne 354 does not teach either system or method for a closed mixing and flushing syringe system.

Within the scope of this application, terms which are absolute, such as round and unreactive, are understood to be permissive of manufacturing and physical limitations which, while functionally achieving a desired function, do not absolutely comply with definition of the specified term.

### BRIEF SUMMARY AND OBJECTS OF THE INVENTION

In brief summary, this novel invention, while having broader applications, alleviates known problems related to providing a normally closed male tapered fitting valve which is opened when a portion of the valve is inserted longitudinally into a tapered female luer fitting. Basic to the instant invention is a valve body having asymmetrical side dimensions formed about a planar slit. The valve body is preferably shaped and sized to conform with the width of the slit providing a predetermined, substantially constant wall thickness from slit to the outer surface of the valve body. The valve body is preferably made from a compliant, incompressible material having memory which, when not inserted, maintains an unconstrained body in its originally formed (e.g. molded) state. Within the valve body are two opposing normally closed slit valves formed about the slit to provide a common fluid pathway when the valves are opened. The valves, being opposed, form a normally closed single valve pathway when uninserted. Generally, the body, being asymmetric, is sized and shaped to reform by compression to fit snugly into a tapered female fitting to, thereby, distort the valve body and open the valve pathway. Of course, for medical applications, the material must also be unreactive to physiologically compatible fluids. Such a material is butyl rubber which is used in contemporary syringe plunger applications.

In Thorne 828, a twisted slit valve is disclosed. Efficacious operation of the twisted slit valve (i.e. closure to fluid flow upon removal from a tapered female fitting) is highly dependent upon valve slit closure due to twisted geometry and molded material interlinking. The instant invention disclosed herein relieves such constraints by providing valve geometry which is designed to be inherently closed when not actuated.

For the case of this instant invention, the two normally closed valves are preferably molded back-to-back within that portion of a device which is inserted into a tapered female fitting. Duckbill valves are particularly well adapted for such purposes. Commonly a duckbill valve has a pair of lips which are closed from pressure in an upstream direction, but open due to pressure exerted in an opposite direction. Placing two duckbill valves such that each valve opposes flow in opposite directions assures valve closure unless the geometry of each valve is physically compromised to open a common communicating pathway.

For a valve which is opened by insertion into a tapered female fitting to operate efficaciously, a number of specific constraints must be overcome. One of the major constraints is associated with circumference compliance. At the distance where the device is fully and sealingly inserted into an associated female fitting, the outer surface of the inserted device should have the same circumference as the internal surface of the tapered female fitting along its inserted length. Also, for compressive forces to accomplish an effective seal, cross sectional area of the inserted fitting plus area of a predefined open pathway should equal the internal cross sectional area of the female tapered fitting along the insertion length. For such a combination to work, the fitting, before being inserted, must be non-circular (asymmetric) yet have the geometric dimensions previously stated.

For small valves, such as valves for luer fittings, dimensional constraints are challenging. To slit a valve while assuring tight maintenance of valve part accuracy, a special technique for valve production and slitting is highly preferred. As an example, inner diameter of a female luer fitting is nominally less the 0.2 inches and lips of a slit valve may need to be less than 0.02 inches thick. To provide parts which can be effectively and efficiently manufactured, a process which molds and slits a valve before displacing critical mold parts away from the mold is preferred. An example of such a method is provided hereafter.

Similar to the twisted valve of Thorne 828, valve opening may be accomplished in either of two modes. The first mode is by compressive distortion of the body to deform the slit from a generally planar state to a more compact hollow cylindrical state, thereby creating an open fluid pathway. As the slit is disposed along a common plane within the valve, a hollow tubular cannula can be displaced through the planar pathway to provide a path for fluid flow, thereby changing the valve to an open state. Note, that, in either case restructuring the body from a compressed state or removing the hollow tubular object results in automatic valve closure.

In the case of valve opening by body distortion, the exterior surface circumference of each body crosscut segment about the slit can be formed to have a predetermined dimension. Likewise, the dimensions of each body crosscut segment will have a predetermined length and width, dependent upon slit length upon which a crosscut circumference conforms. The valve body is preferably designed such that the crosscut circumference is equal along its length to the associated interior surface of a hollow tapered tube (e.g. a female luer fitting) in which the valve is displaced for opening.

In general, a valve device body (e.g. of a male luer adapter), according to the instant invention, has two ends. The first end, comprising the opposed valves, is formed to be used as a fitting element of a tapered releasible connector. The second end comprises a means for forming a communicating, connecting part whereby fluid may be displaced through the valve. If, for example, the slit valve is part of a stand alone male luer adapter, such as those used in common medical applications, the first end would serve as a male luer fitting while the second end may be formed to provide a female luer fitting having a portion which is attachable to a fluid source implement. In such a case, as the male luer fitting portion of the device is inserted into an associated female luer fitting, flexibility of the slit valve allows material to be distorted to conform to the inner circumference of the female luer fitting, resulting in formation of a through hole along the path of the slit and thereby opening the valve. It should be obvious to those skilled in incompressible materials art that the circumference of each cross section of the valve should be of the same dimension as the circumference of the cross section of the associated interior of the luer fitting when the valve is fully inserted into the female fitting to assure a good, sealing fit.

In addition to being used within a stand-alone male luer adapter, using such an asymmetrically formed slit valve as a replacement for a male luer fitting of a syringe provides a basis for closed operation of a plurality of medical syringe applications. Such a replacement provides a syringe which has a closed fluid delivery orifice which remains closed until the male luer adapter is displaced into a female luer fitting, such as a fitting on an IV set or on a medical needle.

In the same manner that the asymmetric slit valve provides closure for a conventional medical syringe, similar use on a syringe which contains barrel disposed valves dividing the syringe barrel into three disparate chambers to yield a closed system for drug mixing and optionally with a follow-on flush. Such a mixing syringe is achieved by employing a first valve which is pressure actuated and resident between two disparate chambers which contain pre-diluted drug in a first chamber and diluent in a second chamber. Mixing is accomplished by applying a force of predetermined magnitude against the barrel and plunger stem to increase pressure within the barrel to open the pressure actuated valve and transfer diluent into the drug containing chamber. Note that, with a pressure actuated valve, the only communication required is force applied between barrel and plunger stem, keeping internal chambers within the syringe closed to an external environment. Fluid may only be transferred from the syringe upon insertion of the male luer valve part into a female luer fitting, thereby keeping system closure intact.

Further, the pressure actuated valve may be fitted with a sensor which senses completed dispensing of the mixed drug and which can then communicate a valve opening trigger to a second valve which is disposed to separate dilution and flush chambers within the barrel and be activated by the trigger. As such, the second valve is preferably displacement actuated. Each valve is designed and sized to maintain fluid in neighboring chambers disparate.

Accordingly, it is a primary object to provide a normally closed valve which is made from a material which is incompressible, elastic and deformable to be compressibly opened when displaced into an elongated, tapered tube having an inside diameter which conformably deforms the valve to open along an imbedded slit.

It is a very important object to provide a normally closed valve comprising but a single molded part which can be affixed as a male luer fitting and opened by displacement into a female luer fitting.

It is an important object to provide a normally closed and self-sealing slit valve which has two distinct and independent opening modes (i.e. by displacement into a hollow tapered tube of predetermined internal surface circumference and by displacement of a blunt cannula through the valve).

It is an object to provide a valve which is displaced to an open state by application of a medially directed force causing compressive deformation.

It is another object to provide a self-sealing valve having a body which is molded from an incompressible, flexible and compliant material.

It is an object to provide a method for making a common slit pathway through two opposing slit valves within a single molded valve body.

It is an object to provide a stand-alone male luer adapter device.

It is another object to provide a normally closed valve which is opened by insertion into a female luer fitting, the valve being formed to be inserted and opened as a male luer fitting for a medical syringe.

It is a very important object to provide a closed syringe system which is normally closed and only opened for fluid flow therefrom by compressive insertion of an integral valved male luer fitting adapter into a female luer fitting.

It is a significant object to provide a closed, single syringe system for disparate containment of drug and diluent and for mixing and dispensing same with an intrinsic separate flush to clear a connecting pathway before disengaging the syringe from the pathway.

It is an object to provide a mixing syringe with only two chambers (i.e. without a flush) but having mixing actuated by a pressure sensitive valved plunger.

These and other objects and features of the present invention will be apparent from the detailed description taken with reference to accompanying drawings.

### BRIEF DESCRIPTION OF THE DRAWINGS

FIG. 1 is a perspective of a male luer adapter device made according to the instant invention.

FIG. 2 is a lengthwise cross section of a distal portion of the device seen in FIG. 1.

FIG. 3 is a lengthwise cross section of the portion of the device seen in FIG. 2, but rotated 90 degrees.

FIG. 4 is a perspective of the male luer adapter device seen in FIG. 1 with three orthogonal planes, each plane being disposed orthogonally across distal parts of selected sections of the device.

FIG. 5 is a cross section of a most distal section of the device seen in FIG. 1.

FIG. 6 is a cross section of a distal section of a medially disposed section of the device seen in FIG. 1.

FIG. 7 is a schematic drawing providing geometry for determining dimensions of a portion of the cross section seen in FIG. 6.

FIG. 8 is a cross section of the section seen in FIG. 5 inserted and fully reformed within a tapered female fitting.

FIG. 9 is a cross section of the section seen in FIG. 6 inserted and fully reformed within a tapered female fitting.

FIG. 10 is a cross section of a most proximal section of the device seen in FIG. 1, the most proximal section being inserted to snugly fit within a tapered female fitting.

FIG. 11 is a lengthwise cross section of a part of a mold used to form the inner or core part of the part seen in FIG. 2.

FIG. 12 is a cross section across a face of a distal end of the part seen in FIG. 11.

FIG. 13 is a lengthwise cross section of the part seen in FIG. 11 with a blunt end tool disposed within an elongated channel of the part.

FIG. 14 is a lengthwise cross section of the part seen in FIG. 11 with a sharp end tool for slitting the sections seen in FIG. 5, the sharp end tool being disposed within the elongated channel of the part.

FIG. 15 is a lengthwise cross section of the part and tool seen in FIG. 13.

FIG. 16 is a lengthwise cross section of a male luer adapter fitting wherein the device seen in FIG. 1 is disposed to form a closed luer fitting.

FIG. 17 is a lengthwise cross section of the male luer adapter fitting seen in FIG. 16, but rotated to show the fitting orthogonally disposed.

FIG. 18 is a lengthwise cross section of a portion of a medical syringe with an embodiment of the device seen in FIG. 1 being substituted for a conventional male luer fitting.

FIG. 19 is a lengthwise cross section of the medical syringe and device seen in FIG. 18 rotated ninety degrees.

FIG. 20 is a cross section of a closed system mixing syringe with integral flush.

FIG. 20A is a cross section of a portion of the system with valve parts seen in FIG. 20, magnified for clarity of presentation.

FIG. 21 is a magnified cross section of the pressure actuated valve seen in FIGS. 20 and 20A.

FIG. 22 is an exploded view of the valve seen in FIG. 21.

FIG. 23 is a magnified cross section of the contact-displaced stem actuated valve seen in FIG. 20.

FIG. 24 is an exploded view of the valve seen in FIG. 23.

FIG. 25 is a cross section of the system seen in FIG. 20 with application of force for opening the pressure actuated valve.

FIG. 26 is a cross section of the pressure actuated valve seen in FIG. 25 at a first opening stage.

FIG. 27 is a cross section of the system seen in FIG. 25 with the pressure actuated valve in a second opening stage.

FIG. 28 is a magnified cross section of the pressure actuated valve in the second opening stage.

FIG. 28A is a cross section of the pressure actuated valve seen in FIG. 28 with pressure as applied in FIG. 25 relieved.

FIG. 29 is a cross section of the system seen in FIG. 27 with diluent transferred to a most distal chamber.

FIG. 30 is a magnified cross section of the pressure actuated valve in contact with the contact-displaced stem actuated valve.

FIG. 31 is a cross section of the system seen in FIG. 29 with diluted and mixed drug dispensed.

FIG. 32 is a magnified cross section of the valves seen in FIG. 31 with the contact-displaced stem actuated valve in a first open stage.

FIG. 33 is a magnified cross section of the valves seen in FIG. 31 with the contact-displaced stem actuated valve in a second open stage.

FIG. 34 is a cross section of the system seen in FIG. 31 with flush dispensed.

FIG. 35 is a cross section of the system seen in FIG. 29 affixed to a gas collection bag and magnified for clarity.

FIG. 36 is a further magnified partial cross section of the gas collection bag seen in FIG. 35.

FIG. 37 is a cross section of a portion of the mixing system seen in FIG. 20A with parts associated with flushing removed.

FIG. 38 is a perspective of an internal support for the device seen in FIG. 1.

FIG. 39 is a proximal elevation of the support seen in FIG. 38.

FIG. 40 is a cross section of the support seen in FIG. 38 disposed in the device seen in FIGS. 1 and 16.

FIG. 41 is a cross section of the parts seen in FIG. 40 rotated 90 degrees.

FIG. 42 is a perspective of an exemplary mold for the device seen in FIG. 1.

FIG. 43 is an exploded perspective of the mold seen in FIG. 42.

### DETAILED DESCRIPTION OF THE ILLUSTRATED EMBODIMENTS

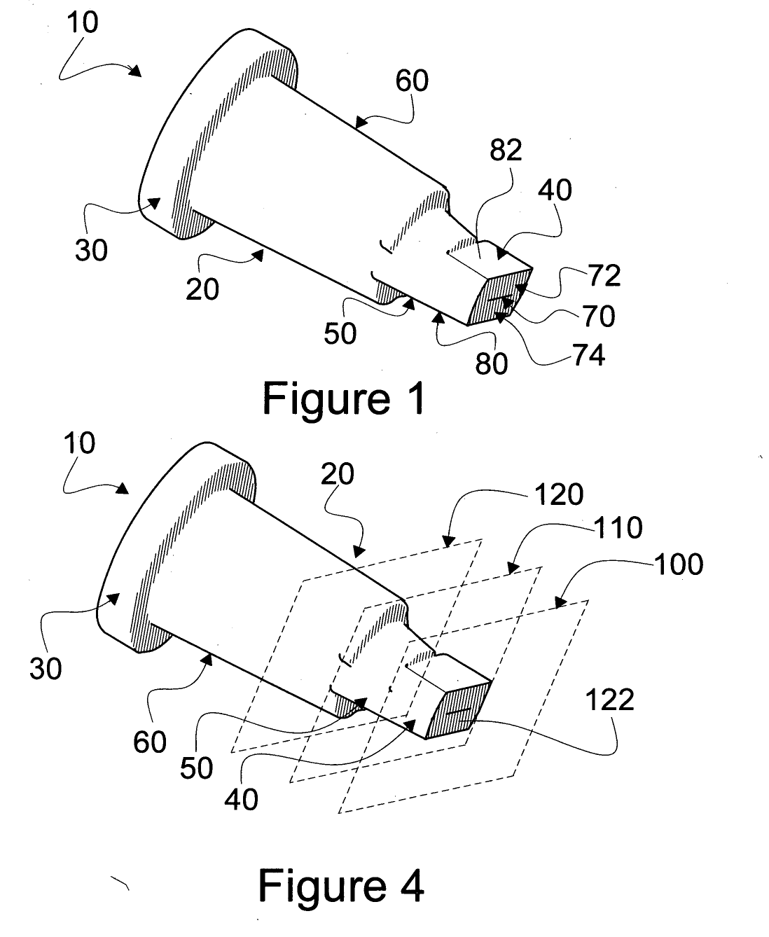

While the instant invention disclosed herein is applicable to a wide variety of tapered male/female insertion type fluid connectors, the detailed description provided herein is focused upon medical luer fittings and luer fitting applications. In this description, the term proximal is used to indicate the segment of the device which is a closest part to an object of reference. The term distal refers to the opposite orientation. Reference is now made to the embodiments illustrated in FIGS. 1-43 wherein like numerals are used to designate like parts throughout and primes of numbers generally indicate parts which are similar in shape and/or function of those numbers, but not exactly the same.

Seen in FIG. 1 is a device 10 made according to the instant invention and which exemplifies the presently preferred embodiment. Device 10 is seen to comprise two segments, a luer fitting segment 20 and an attachment ring 30. Segment 20, also referenced hereafter as fitting 20, generally comprises the following three parts disclosed relative to an attachment, such, as attachment ring 30:

1. A distally disposed first valve section 40

2. A medially disposed second valve section 50 and

3. A proximally disposed tapered section 60.

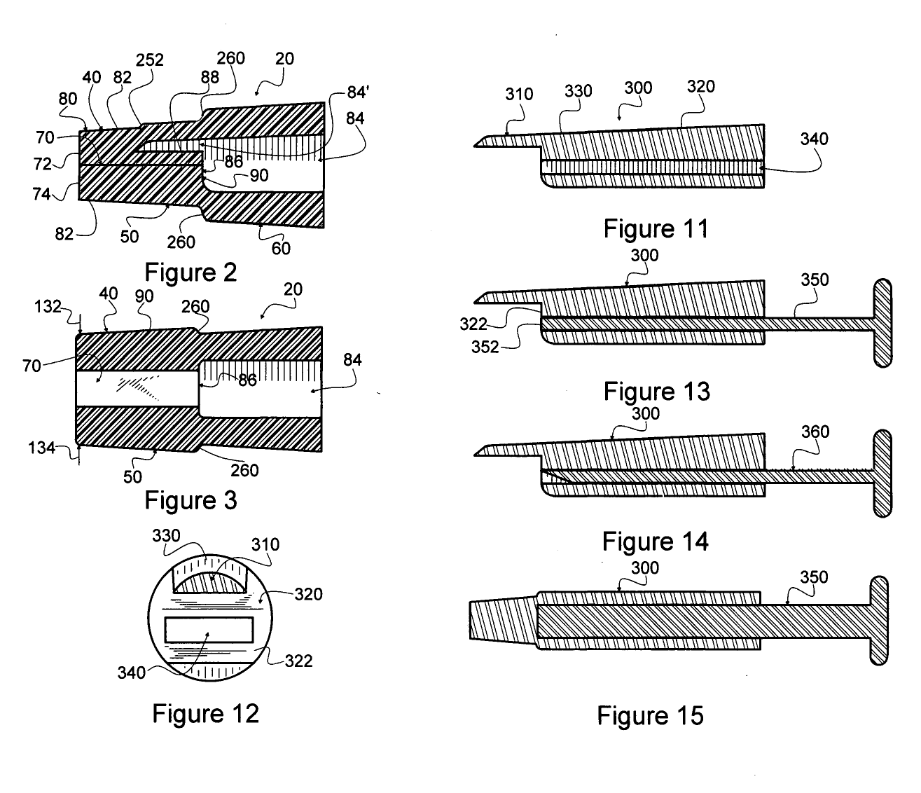

Sections 40, 50 and 60 are seen in cross section in FIGS. 2 and 3. Attachment segment 30 (see FIG. 1) is not seen in FIGS. 2 and 3 as sections 40-60 are common to all luer applications and form and function of device 10 attachments may be changed for various applications, examples of which are disclosed in detail hereafter. A slit 70 through section 40, seen in FIGS. 1 and 2, forms a pair of lips, numbered 72 and 74, forming a first normally closed valve 80. Valve 80 is similar to a duckbill valve and is generally closed by pressure greater on an exterior surface 82 of fitting 20 than is present in an interior cavity 84 (see FIGS. 1 and 2) formed within fitting 20.

Also, as seen in FIGS. 1 and 2, slit 70 is contiguous through section 50 forming a second pair of lips 86 and 88, which are parts of a second normally closed valve 90. Note that a portion of cavity 84 is extended distally to provide a smaller cavity 84′ which further provides a fluid pathway in communication with lip 88. Cavity 84′ serves as a fluid containing volume wherein a higher than ambient pressure can be applied upon lip 88. By applying a higher than ambient pressure, such as pressure applied via a syringe plunger, lip 88 is compressively engaged against lip 86 to provide a seal which closes valve 90. In this manner, valves 80 and 90 are opposing, normally closed valves and cannot be opened by a pressure differential imposed in either direction. The only way to open the opposing, normally closed valves is by compressively forcing lips 72 and 88 apart from lips 74 and 86, respectively. Such is accomplished by forming sections 40 and 50 as asymmetric components having dimensions which are compressively reformed when displaced into a tapered fitting, such as a female luer fitting, to force lips 88 and 86 and 72 and 74 apart.

To be effective, device 10 should be made from pliant, incompressible material such as butyl rubber or silicone. Of course, for medical applications, such material should be biomedically inert. As well, for efficacious operation, the asymmetric form of sections 40 and 50 should be shaped and dimensioned such that when compressed to snugly fit into a female fitting no sidewall leakage occurs during fluid transmission. For this condition to be met, at a depth of full insertion, each section 40 and 50 should have the same surface dimensions (orthogonal circumferential lengths) and fill the same cross sectional areas as the connected female luer fitting. When these conditions are met, an inserted device 10 fulfills all of the connecting requirements for a conventional male luer fitting. As well, when device 10 is not so interconnected, valves 80 and 90 should revert to original molded shape and are closed to fluid flow.

Reference is now made to FIG. 4 wherein device 10 is seen accompanied by three planes, numbered 100, 110 and 120, each of which is disposed to orthogonally cross cut the most distal part of sections 40, 50 and 60 of fitting 20, respectively. Section 40, being most distal from attachment ring 30, is of smallest dimension due to taper of fitting 20, which is compatible with a 3 degree luer taper. While a luer fitting made according to the instant invention may have a facilely insertable size for open valve fluid delivery holes which range from 0.040 to 0.065 inches, for purposes of example a through hole diameter of 0.045 has been selected for exemplary disclosure herein. Those who are skilled in trigonometry and luer fitting design should be able to extrapolate to designs for different hole sizes from information provided hereafter.

### Considerations for Section 40, Distal Valve 80

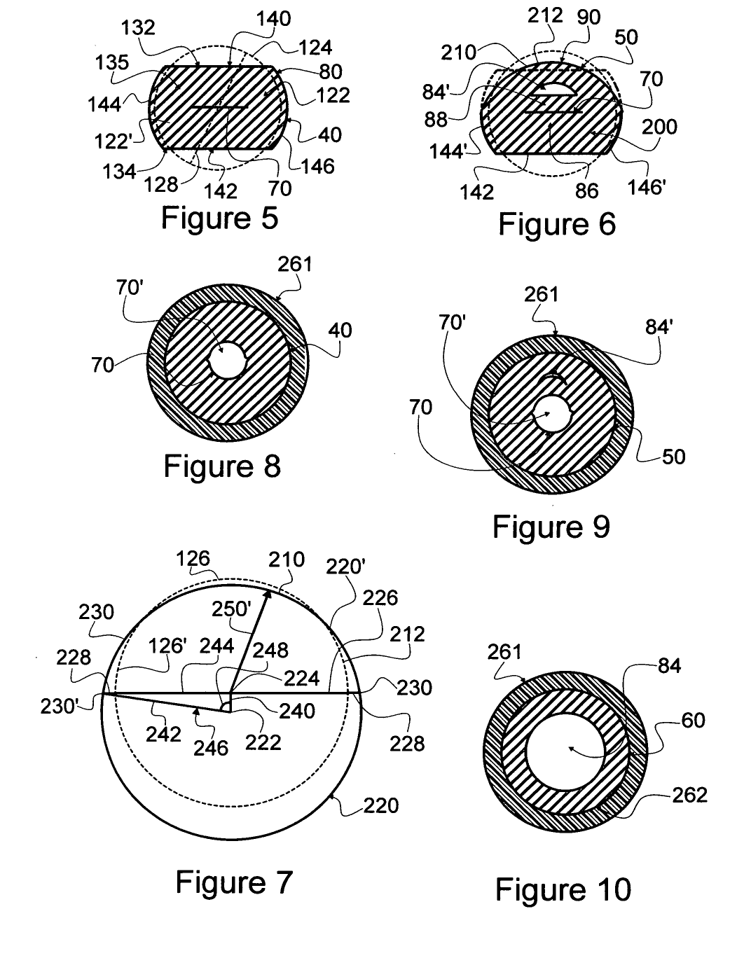

A cross section of the distal end 122 of section 40 is seen in FIG. 5. For reference, a representation of the inner circumference 124 of a luer fitting surface at the site of deepest insertion of fitting 20 is seen as a dashed line 126. At that depth, a diameter (Dₚ), referenced by dashed line 128, of an associated female fitting, is calculated to be 0.143 inches. A medially disposed slit 70 for a 0.045 inch pathway, hole diameter (Dₕ), has a slit length (Lₐ) of approximately 0.071 inches. An arbitrary, but considered useful condition established for this example is that the sidewall about the fluid pathways of the valves are of uniform material thickness. Based upon this condition, the thickness (T_{f}) of face 122′ (i.e. the perpendicular distance between arrows 132 and 134, see FIG. 3) is provided by the following equation:

T_{f}=Dₚ−Dₕ

Thus, thickness of a so defined sidewall 135 (see FIG. 5) is ½ T_{f}.

Face 122′ is bounded by two parallel surfaces 140 and 142 and two opposing rounded side surfaces 144 and 146. In this example, side surfaces 144 and 146 have the same radius of curvature as dashed line 128 (which is the radius of curvature of circumference 124). The longest distance between side surfaces 144 and 146 is defined to be the face length (L_{f}) which is defined as follows:

L_{f}=2T_{f}+Lₛ

Note that L_{f} extends surfaces 144 and 146 outward beyond circular dashed line 124, representing circumference of the inner surface of a female luer fitting at that insertion depth. The length of outward extension beyond line 124 is equal to ½ (Lₛ−Dₕ) or the distance each face 122 and 122′ must be compressed to open a hole defined by slit 70. Note also that the radial distance between each surface 140 and 142 and dashed line 124 is ½ hole diameter Dₕ. Face length at each face 122 and 122′ is approximately 0.169 inches for a fitting designed to be inserted into a conventional female luer fitting to a depth of 0.330 inches. Such a face of device 10 can easily be inserted into a conventional luer fitting which is nominally 0.178 inches in diameter at a proximal opening thereof. As device 10, in this example, is specified for a luer fitting, all dimensions except for hole diameter Dₕ and slit length Lₛ, are increased linearly to compensate for a 3% taper. Valve length along an elongated medial axis of device 10 may vary depending upon device 10 material characteristics such as durometer. In this example, the length of valve 80 is 0.10 inches. It may be noted that duckbill valves commonly have a thickness of 0.050 inches so consideration may be given to a shortening of the length of valve 80. In this example, valve 90, disclosed hereafter, has a similar length to valve 80 similar shortening considerations apply.

### Considerations for Section 50, the Proximal Valve 90

Referring again to FIG. 4, a cross cut at plane 110 provides a view of the most distal face 200 of section 50 (see FIG. 6). As seen in FIG. 6, distal face 200 comprises lower lip 86, slit 70, upper lip 88 and cavity 84′. Face 200 is bounded by surfaces 142, 144′, 146′ and 210. Surfaces 144′ and 146′ only extend from surface 142 to height of slit 70. Surface 142 is planar while surfaces 144′ and 146′ have the same relative curvature as surfaces 144 and 146 (see FIG. 5). Surfaces 144′ and 146′ have only half the surface dimension of surfaces 144 and 146 as only the inferior half of section 40 is continued through section 50. To permit inclusion of cavity 84′, the most superior side 212 of section 50 is arched. Surface 210 must have the same cross sectional circumference of an associated female luer fitting when disposed therein for reasons given supra. For ease of calculation, an arc of a circular surface has been selected for surface 210.

A schematic diagram of edges of surfaces 210 and circumference 126 of an inner surface 212 of an associated female luer is seen in FIG. 7. As seen in FIG. 7, surface 210 can be represented by an arc of a circle 220. A center point 222 of circle 220 is offset from center point 224 of the circle of inner circumference 126. A width of inner surface 212 at plane 110 (see FIG. 4) is represented by line 226. As disclosed supra, the length of outward extension beyond line 126 (see FIG. 5) is equal to ½ (Lₛ−Dₕ). This same outward extension applies to section 50. Thus, the outward extension beyond diameter 128 is also ½ (Lₛ−Dₕ). Such outward extensions are represented by line segments (commonly numbered 228), on each side of line 226. Outward end-points of segments are numbered 230 and 230′. To meet established criteria for surface circumference at each plane, an arc 220′ of circle 220 must intersect points 230 and 230′ and be of the same circumferential length as a half-circle of inner circumference 126.

Length and curvature of arc 220′ are calculated as follows: A line 240 represents offset distance between center points 222 and 224. A line 242 represents the radius of circle 220 (and arc 220′). A line 244 represents radius of inner circumference 126 plus length of offset line segment 228. Lines 240, 242 and 244 construct a right angle triangle 246. An angle 248 is disposed opposite line 244. Arc length of one-half inner circumference 126 is pi (3.1415926) times the length of inner circumference radius 250. Sine of angle 248 equals length of line 244 divided by length of line 242. Angle 248 is the arc sine (in degrees) of sine of angle 248. The length of inner circumference line 126′ is pi time radius 250 which is the desired length of arc 220′. Length of arc 220′ is equal to pi times radius 242 times 2*angle 248 divided by 180. Of course, radius 242 may not be simply mathematically determined. However, employing a computer based number processing system, such as MicroSoft Excel®, a reasonable guess of length of radius 242 with comparison of calculated arc length against length 220′ permits an iterative process for solving for radius 242. For a 0.045 inch hole, radius 242 is approximately 0.09444 inches.

### Considerations for Section 60

Referring once more to FIG. 4, a cross cut at plane 120 provides a view of the most distal face of section 60. As seen in FIG. 2, section 60 has conventional dimensions for a luer fitting disposed between section 50 and an attachment, such as segment 30. Presently preferred length of section 60 is 0.300 inches.

### Interfaces Between Sections

As a consequence of the various designs of sections 40-60, a discontinuity occurs between proximal and distal faces of sections 40 and 50 and 50 and 60. Discontinuities are exemplarily represented by dashed lines in FIG. 6. For this reason, as seen in FIGS. 2 and three, it is such discontinuities (i.e. designated by number 252 between section 40 and section 50 and by number 260 between section 50 and 60) be smoothed by filleting. It should be kept in mind that surfaces of the discontinuities should have the same circumferential constraints as those of section 40-60.

Form of Segment 20 after Insertion into a Female Luer Fitting

As stated supra, insertion of segment 20 into a female luer fitting (generally numbered 261) results in a medially directed compression of inserted sections of device 10 and a change in shape for section 40 as seen in FIG. 8. Slit 70 has been opened to provide a hole 70′.

Similarly, compression of inserted section 50, as seen in FIG. 9, results in opening slit 70 to a hole 70′ having substantially the same dimensions as hole 70′ in section 40 (see FIG. 8). Note that cavity 84′ is diminished in volume by material displacement which opens hole 70′.

Reference is now made to FIG. 10. As the outer surface 262 of section 60 is sized and shaped to conform as a conventional male luer fitting within a female luer fitting, there is little change to cavity 84 and displacement of surface 262.

Making Device 10

Reference is now made to FIG. 11 wherein a tool 300 used to mold and slit device 10 is seen. Tool 300 (also referenced as core and slitter 300) is used in conjunction with an outer mold which forms outer surfaces of device 10. As device 10 may be formed by numerous ways using well known technology to those skilled in the art of making butyl rubber and silicone parts, the outer portion of a mold used to form device 10 will not be further dealt with herein, except for molding and slitting considerations disclosed hereafter.

Tool 300 has attributes which form cavity 84 and cavity 84′. Those attributes include a distal tongue portion 310 and a larger proximal portion 320. Tool 300 is preferably made from stainless steel and has a distal face 322, seen in FIG. 12. A superior surface 330 is flattened to provide continuity to tongue 310, as seen in FIG. 11 This flattening permits a greater amount of material be provided to surround and thicken the surrounding wall of device 10 about cavities 84 and 84′. (see FIG. 2) Of constant concern when making device 10 is the size of the tapered fitting being formulated. As an example, diameter of portion 320 is preferably about 0.1 inches in diameter.

Tool 300 also has an elongated open channel 340 through which a slitting knife can be displaced to slit sections 40 and 50 (See FIG. 2). During the molding process, a tool 350, which is sized and shaped to be displaced through channel 340, is disposed within channel 340 to inhibit material being displaced into channel 340 during molding as seen in FIG. 13 and seen rotated 90° FIG. 15. For this purpose, tool 350 has a blunt end 352. As there is only 0.05 inches available for lip 88 (formed by slitting), width of tongue 310 (which also, of fabrication necessity, should be about 0.020 inches thick, see FIG. 11) and desired thickness of material surrounding tongue, tolerances and precision of slitting must be carefully maintained. For this reason, channel 340 for a slitting knife is made part of the mold to overcome variances which can occur when a slitting operation is kept disparate from the molding operation.

Following molding, tool 350 is removed from channel 340 and replaced by slitting tool 360. See FIG. 14. Also, because material from which device 10 is made is essentially incompressible, mold parts which surround at least inferiorly disposed parts of sections 40 and 50 should be removed before slitting. Slitting should be performed in a single step of displacing slitting tool 360 through sections 40 and 50 to form slit 70. It should be noted that since tool 300 is a core of an associated mold for device 10, size and shape of portion 320 and tongue 310 are complimentary to cavities 84 and 84′, respectively.

### EXAMPLES OF DEVICES 10 AND 10′ APPLICATIONS

### Example 1

### As a Normally Closed Valve in a Male Luer Adapter

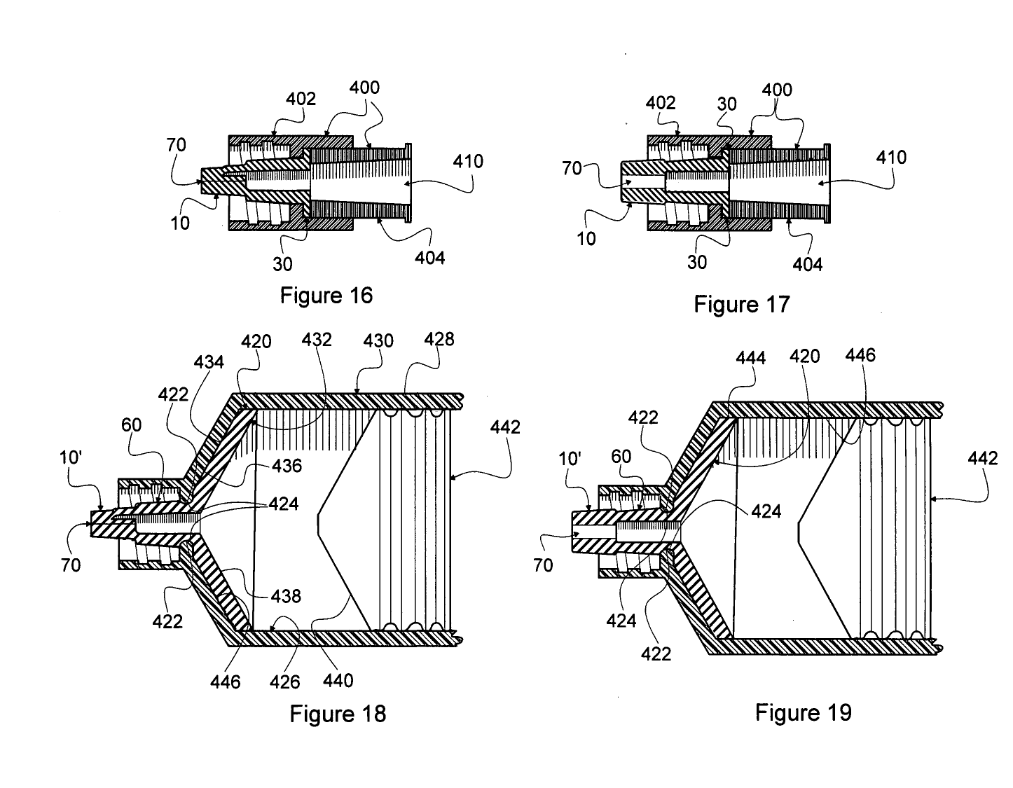

Referring to FIGS. 16 and 17, device 10 is seen encased into a housing 400 formed by a luer lock part 402 and a female luer fitting 404. Device 10 has an attachment ring 30 which is sandwiched between parts 402 and 404 to isolate a fluid path 410. Device 10 substitutes for a male lure fitting in luer type connections providing a fitting which is closed when disposed as seen in FIGS. 16 and 17, but opened when displaced into a female luer fitting to function as a stand-alone male luer adapter.

### Example 2

### As a Closed Valve Replacement for a Male Luer Fitting of a Syringe

In FIGS. 18 and 19, attachment ring 30 is replaced with a syringe attachment appendage 420 to form a device 10′. In particular appendage 420 has an annular groove 422, proximally disposed relative to section 60, which snugly fits into a molded collar 424 in an otherwise conventional inner portion 426 of a barrel 428 of syringe 430. Groove 422 and collar 424 act to provide a fluid seal between device 10′ and syringe 430. A more proximal concave frustoconical part 432 of appendage 420 has a distal face 434 which conforms with the concave inner distal surface 436 of syringe 430. Appendage 420 has a proximal face 438 which conforms in shape and size with a face 440 of an associated syringe plunger 442. To minimize dead space, size and shape of the exterior surface 444 of appendage 420, appendage 420 snugly fits into the inner surface 446 of syringe 430.

### Example 3

### As an Integral Part of a Closed System Mixing Syringe with Flush

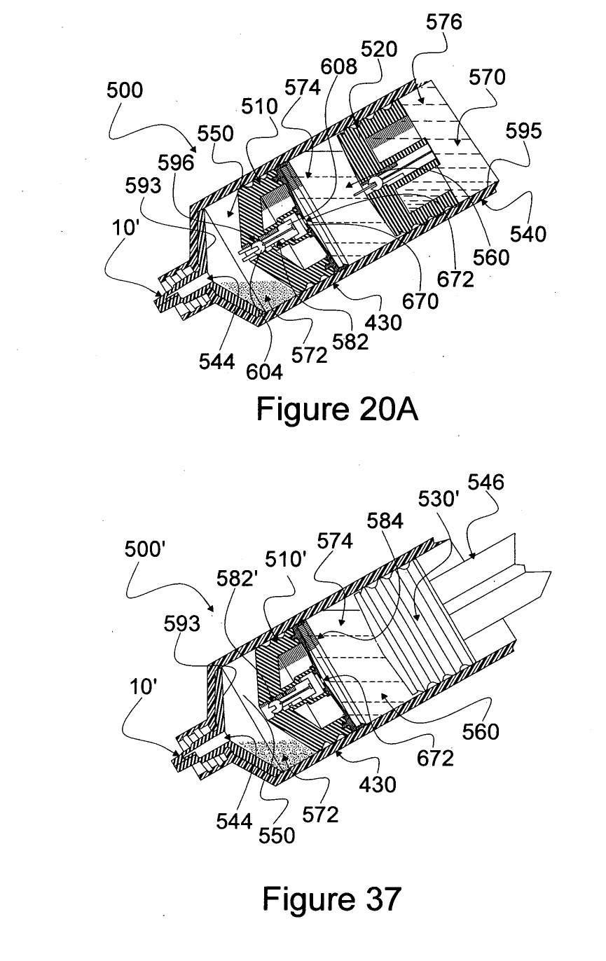

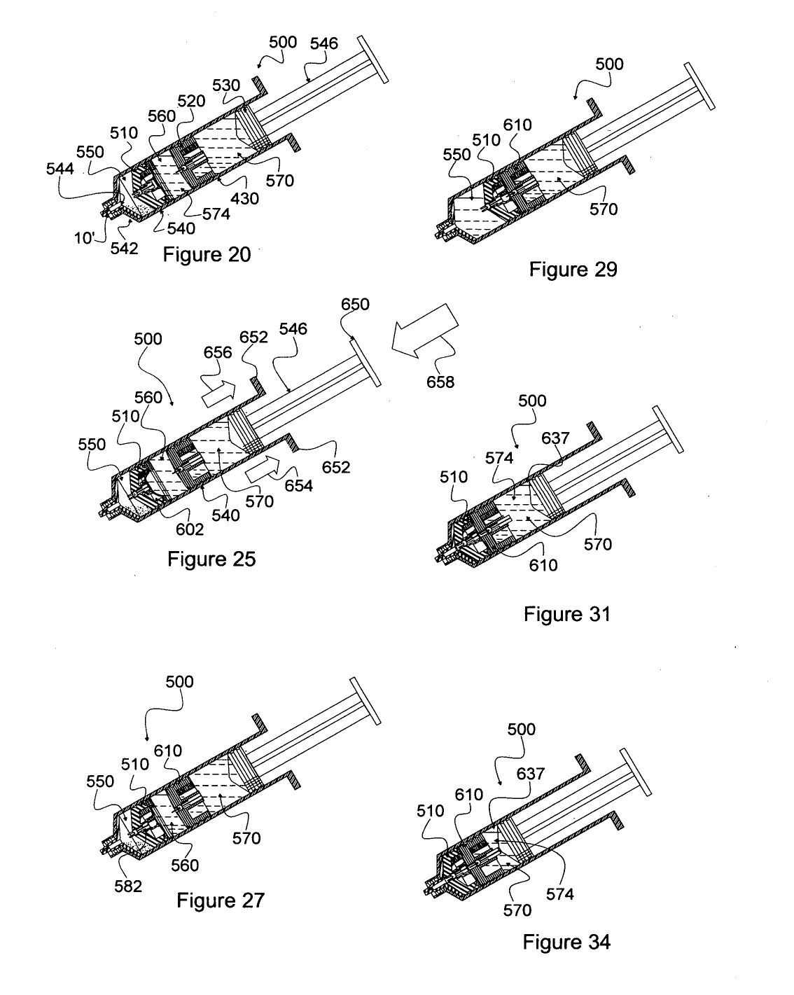

Reference is now made to FIGS. 20-37 wherein various operational states and attributes of a closed system mixing syringe are seen. Referring to FIG. 20, a mixing syringe with flush 500 is seen to comprise a medical syringe 430, a device 10′ (see FIG. 19) and other parts which act cooperatively to provide a mixing syringe with flush. The other parts comprise a distal valved plunger 510, a medial valved plunger 520 and a proximal plunger 530. Syringe 430, with the exception of accommodating device 10′, which provides a closed valve fitting (fitting segment 20, see FIG. 1), is conventionally made with a barrel 540 diminished at a distal end 542 to provide an orifice 544 (see also FIG. 20A) through which fluid is communicated. Device 10′ is disposed to close flow through orifice 544 until actuated to an open state by inserting device 10′ into a female luer fitting (see FIG. 9). Proximal plunger 530 and an associated stem 546 (see again FIG. 20) are conventional syringe parts used to displace fluid withing barrel 540. Stem 546 may be a threaded part for insertion into plunger 530 after liquid is stored in syringe chambers.

More easily seen in FIG. 20A, distal valved plunger 510 is sized and shaped to displace fluid within barrel 540 while in a normally closed state. So disposed, plunger 510 provides a fluid separating barrier within barrel 540 forming a distal chamber 550. Medial valved plunger 520 is also sized and shaped to displace fluid within barrel 540 while in a normally closed state and is disposed proximally from plunger 510 to form a medial chamber 560 disposed between valved plungers 510 and 520. A most proximal chamber 570 is formed by separation of plungers 520 and 530 (see FIG. 20).

Generally, in a mixing application, a drug (e.g. lyophilized drug 572 (as seen in FIG. 20A) may be disposed in chamber 550 and a diluent 574 may be disposed and stored in chamber 560. A flushing agent 576 may be stored in chamber 570. For storage and transport camber contained materials are maintained disparate by valved plungers retained in a closed state.

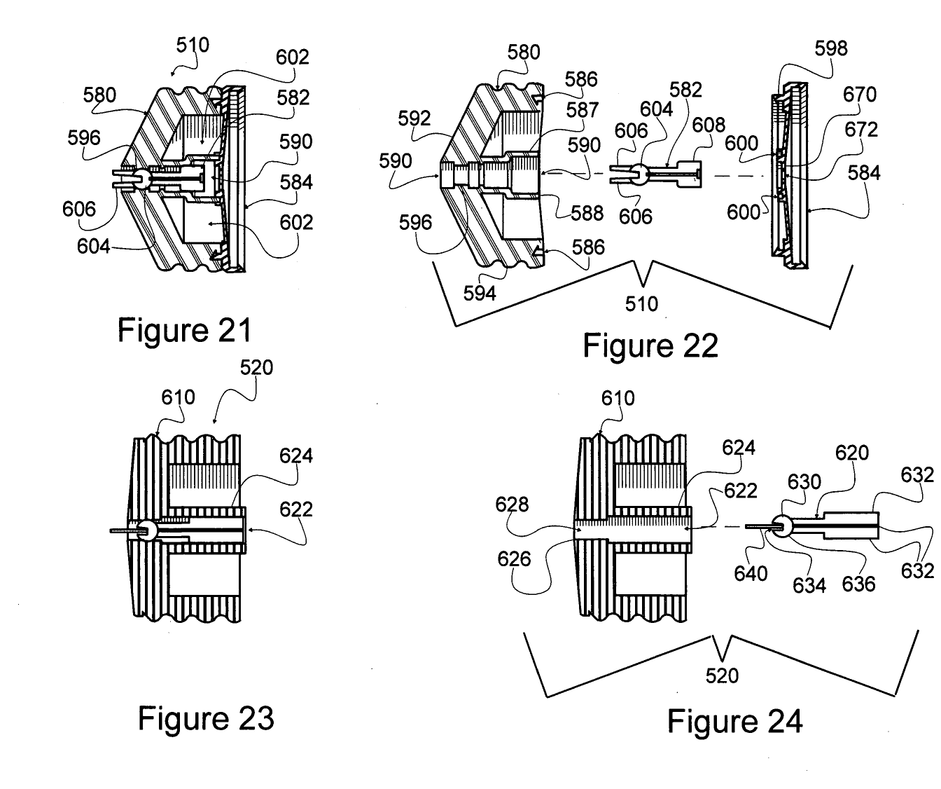

Valved plunger 510, seen in FIGS. 21 and 22, comprises three parts, a substantially rigid housing 580, a valve actuating stem 582 and an elastically compressible housing plate 584. Housing 580 comprises a circular connecting groove 586 (see FIG. 22) and a cylindrical center part 587 comprising a circular rim 588 disposed about a cylindrical fluid passageway 590. Housing 580 is preferably made from an incompressible, human fluid compatible, elastic and flexible material such as butyl rubber (a common material used in syringe plungers). Additionally housing 580 has a distally disposed surface 592, which is made to conform in size and shape with a contacting distal concave frustoconical inner surface 593 of device 10′ (see FIG. 20A) if an associated syringe contains a device 10′ and with a proximally disposed concave surface of a conventional associated syringe should a device 10′ not be employed. Further, housing 580 has side walls 594 which are sized and shaped to displace fluid along an inner side wall 595 (again see FIG. 20A) of barrel 540 in the same manner as a conventional plunger. Additionally, housing 580 has a narrowed portion 596 of passageway 590, the purpose for which is fully disclosed hereafter.

Housing plate 584 is preferably made from the same material as housing 580 and comprises a circular attachment ring 598 and a circular attachment groove 600. Ring 598 and groove 600 being complimentary in size and shape to groove 586 and rim 588, respectively, such that the rings and grooves provide attachable and sealable connections between housing 580 and plate 584. In assembly, housing 580 and plate 584 are sealed together, preferably either by an effective adhesive or ultrasound welding to sealingly enclose a chamber 602 (see assembled valved plunger 510 in FIG. 21) disposed about passageway 590. Sealed chamber 602 must contain a compressible fluid (e.g. air) at a nominal pressure (e.g. atmospheric, at time of sealing).

Referring again to FIG. 22, valve actuating stem 582 comprises a medially disposed bulbous portion 604, sensor tines (commonly numbered 606) and a proximally disposed winged portion 608. Bulbous portion 604 is sized and shaped to be tightly seized and thereby close fluid flow at narrowed portion 596 when disposed therein. It is important to note that the seizing force associated with bulbous portion 604 disposed within narrowed portion 596 must be greater than any force associated with force of pressure of fluid displacing plunger 510 through barrel 540. If such is not the case, an accidental application of force against could cause displacement of stem 582 and inadvertent communication of fluid from chamber 560 to chamber 550. Likelihood of such happening in syringe 500 not of significant concern, however, because of the relative pressure affecting area of bulbous portion 604 compared to area of plate 584 and differential pressures between chambers 550 and 560 and the requirement that opening valved plunger requires force due to a higher pressure than a differential pressure required to displace plunger 570. This difference provides a significant margin of safety not found in the construct of a similarly applied valve disclosed in Thorne 354, wherein a one-way duckbill-like valve is disclosed which cannot withstand a force less than the force required to displace a valve plunger without opening for fluid transmission. Additional attributes and characteristics of housing 580, stem 582 and plate 584 are disclosed hereafter relative to valve actuation and operation. Stem 582 is preferably made by injection molding from the same material used to make barrel 540.

As seen in FIGS. 23 and 24, valved plunger 520 comprises two parts, body 610 and stem 620, body 610 and stem 620 being made from the same materials as housing 580 and stem 582, respectively. With the exception of design variations disclosed hereinafter, valved plunger 520 is similar in construction and operation to the valve disclosed as “discharge assembly 70” for sequential delivery syringes in U.S. Pat. No. 7,789,862 B2 titled MULTI-CHAMBER, SEQUENTIALLY DISPENSING SYRINGE issued to Gale H. Thorne, Jr. Sep. 7, 2010.

As seen in FIG. 24, body 610 has a medially disposed channel 622 disposed within an elongated cylindrical tube 624, with channel 622 being constricted at a distal end 626 to form a smaller orifice and channel 628. Valve stem 620 has a bulbous portion 630 which is sized and shaped to close channel 628 to fluid flow when disposed therein. Also, stem 620 has a set of stabilizing wings (commonly numbered 632) which are generally sized and shaped to glide within channel 622. Valve stem 620 has a duct 634, distally disposed from a solid hemispherical section 636, through which fluid flows as bulbous portion 630 is displaced from channel 628. In addition, stem 620 has a rigid sensor element 640 against which a proximally displaced solid facing causes proximal displacement of stem 620 for displacing stem 620 to open valved plunger 520. Further, elongated tube 624 has sufficient length to extend into a “liquid only zone” whereby only liquid is permitted to flow from chamber 570 when valved plunger 520 is open (see FIG. 20A). An air pocket 637 is seen disposed, trapped and unable to be dispensed from chamber 570 (see FIGS. 31 and 34). Additional features and structure for valved plunger 520 are disclosed hereinafter.

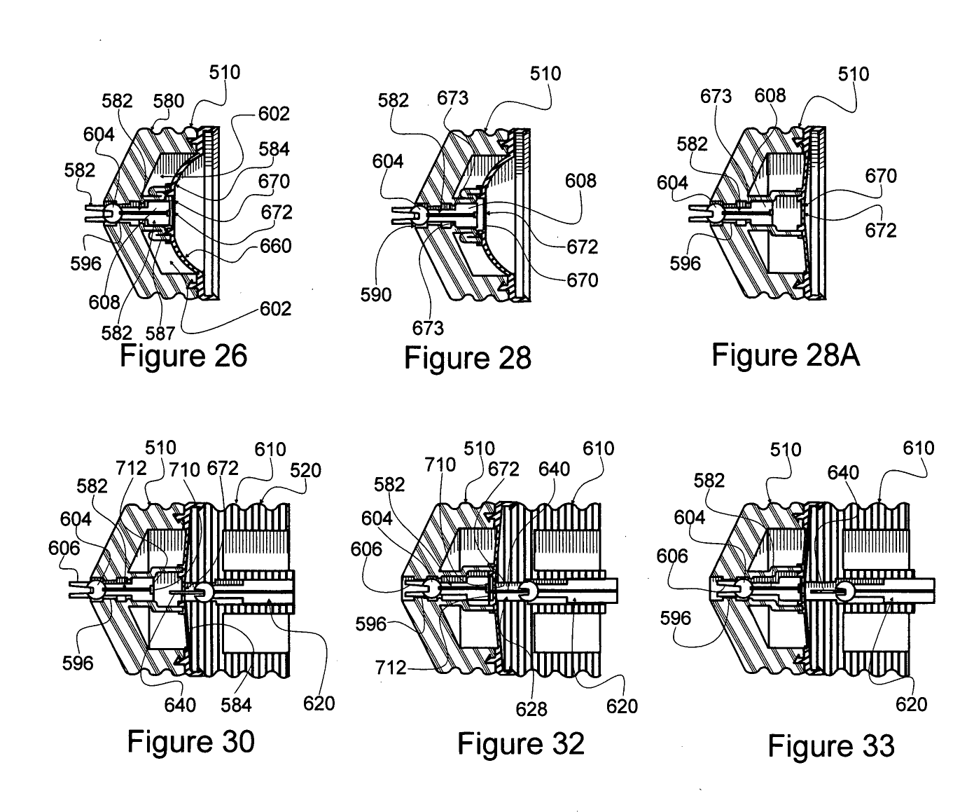

To initiate a mixing step, valved plunger 510 must be opened to permit displacement of diluent 574 from chamber 560 into chamber 550 (see FIG. 25). At mixing step initiation, force is applied against plunger stem 546 and barrel 540 via stem actuator 650 and barrel flanges (commonly numbered 652) in directions of indicating arrows 654, 656 and 658 to increase fluid pressure within barrel 540. Increased pressure about chamber 602 causes gas compression and resulting volume reduction. As seen in FIG. 26, plate 584, being thinner in construction and, therefore, more easily compressively displaceable than housing 580 is stretched and forced inward as gas in chamber 602 is compressed. Notice stretching of wall 660 of plate 584. As wall 660 is stretched and forced inward, tubular cylindrical central part 587 is pulled outward and subjected to a folded state. Plate 584 is seen to have a circular collar 670 formed about access hole 672. Collar 670 and access hole 672 are better seen in FIG. 28A. As plate 584 is forced inward, collar 670 contacts wing 608 to force stem 582 distally until bulbous portion 604 is more than half-way displaced from narrowed portion 596. Compressive forces associated with plunger compression within barrel 430 then completes displacement of bulbous portion 604 from narrowed portion 596, as seen in FIG. 28. Once pressure delivering force is removed, valved plunger 510 returns to original shape with stem 582 displaced to provide and open state for valved plunger 510 as seen in FIG. 28A.

While it is not necessary to provide a one-way valve to pump and displace fluid 574 from chamber 560 into chamber 550, a one-way valve significantly improves pumping efficiency. Bulbous portion 604 of stem 582 and narrowed portion 596 combine to provide one-way flow control due to a restraining ring 673 (see FIGS. 28 and 28A), disposed within channel 590 to act against wings 608 to responsively bias bulbous portion 604 against narrowed portion 596 when pressure in chamber 550 (see FIG. 20A) is equal to or less than pressure in chamber 560.

With a one-way valve disposed between chambers 550 and 560, diluent 574 and any other fluid is displaced into chamber 550 by once again applying force as indicated by arrows 654, 656 and 658 (see FIG. 25) in a sequential pumping action until all fluid is displaced from chamber 560. Plungers 510 and 520 are seen disposed with fluid so removed from chamber 560 in FIG. 30. Important to note is that plate 584 comprises a centrally disposed hole 672 (see FIGS. 20A and 30). Note also, as seen in FIG. 30, that, with stem 582 proximally disposed, there is no contact between stem 582 and stem 620 as elongated sensor element 640 of stem 620 is displaced through hole 672. It is very important that element 640 (see FIGS. 24 and 32) be sized and shaped to be displaced through hole 680 without contacting plate 584.

Referring to FIGS. 27 and 29, valved plunger 510 is disposed in an open state after distal displacement of stem 582, as more readily seen in FIG. 28A. Chamber 560 is seen emptied into chamber 550 in FIG. 29.

Mixing occurs as fluid is dispensed into chamber but sufficient time and swirling of mixed contents according to drug protocol should occur before a dose is ready to be dispensed. As contents of chamber 550 likely contains both liquid and gas matter, as is well known in IV delivery to a patient, gas must be expelled from chamber 550 before dispensing to a patient.

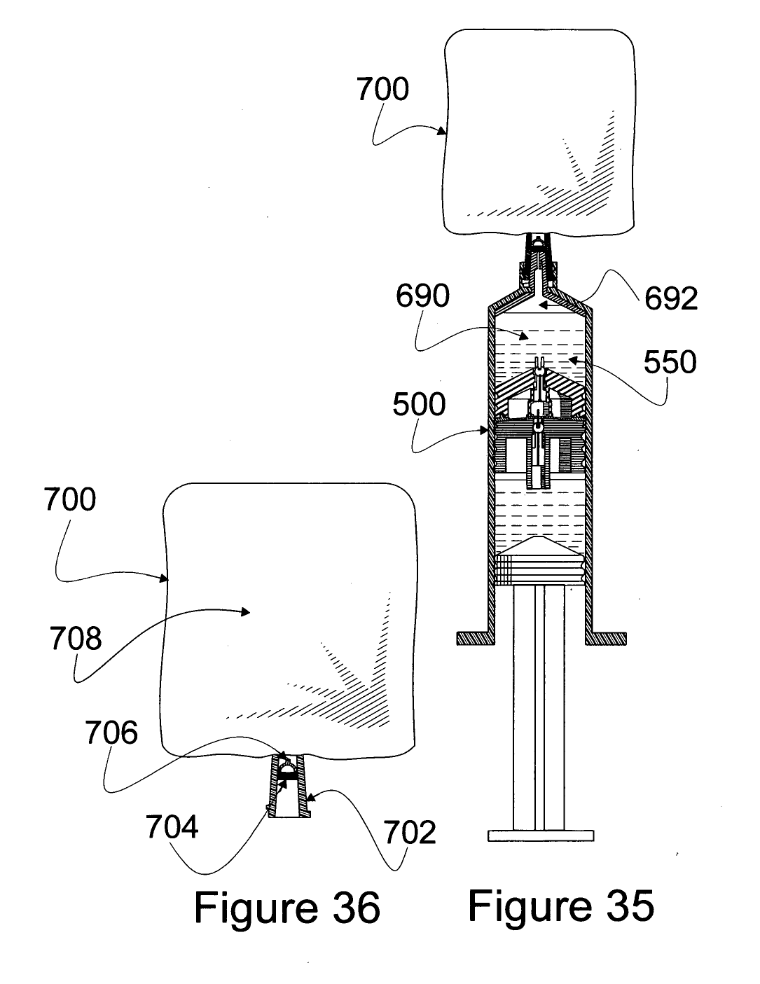

It is common practice to simply expel gas from a syringe by holding the syringe upright and dispel gas to the open environment. However, for hazardous drug handling such is considered unsafe as it violates closed system protocol. It is preferred to expel gas to a contained environment to preserve closed operation. For this reason, it is preferable to expel gas from chamber 550 as seen in FIG. 35. Note that chamber 550 contains a fluid comprising liquid 690 and gas 692. All such fluid remains captive in chamber 550 until device 10′ is opened. As seen in FIG. 36, a closed waste bag 700 can be used to dispose of unwanted, contaminated gas 692. Preferably, as noted in FIG. 36, waste bag 700 comprises a female luer connector 702 with a hydrophobic filter 704 which passes gas, but not liquid and a one-way (e.g. a duckbill) valve 706 all of which provide for receipt and capture of gas from chamber 550. Device 10′ is opened when displaced into luer fitting 702 and self-closes when removed therefrom.

Once gas is removed from chamber 550 mixed contents of chamber 550 can be completely dispensed as seen in FIG. 31. Of course, all contents of chamber 550 should be dispensed before a flush is delivered. This is accomplished by action of stem 582 in combination with narrowed portion 596 of plunger 510 (see FIGS. 30 and 32). Sensor tines 606 of stem 582 are sized and shaped to sense a collision with device 10′ at orifice 544 (see FIG. 20A) or a dispensing orifice if a conventional syringe is employed. As plunger 510 is displaced distally after such a collision, plunger 510 continues to be displaced proximally relative to stem 582. First displacement of plunger 510 causes bulbous portion 604 to be resident within narrowed portion 596 once again, closing valved plunger 510 and assuring dispensing of contents of chamber 550 (see FIG. 31). As plunger 510 is further proximally displaced about bulbous portion 604, as seen in FIG. 32, valved plunger 510 is again opened. Also, a target plate 710 (see FIGS. 30 and 32) medially disposed at the proximal end 712 of stem 582 in combination with sensor element 640 of stem 620 communicate to proximally displace stem 620 from channel 628 (see FIG. 24) to open valved plunger 610 as seen in FIGS. 31 and 33. Opening valved plunger 610 in combination with an open valved plunger 510 permits dispensing of flushing agent 574 (see FIG. 37) to wash through all connecting lines as a guard against exposing an open connecting site to the surrounding environment.

There are instances where a flush is not required of a mixing syringe, such as, when syringe protocol instructs leaving a spent syringe on an IV line. Also, no flush may be required if a given drug is not considered hazardous. In such cases, a mixing syringe may be made without an associated flush eliminating the need for one chamber and one valved plunger within the mixing syringe and resulting in a simpler, lower cost system.

Such a system is seen as mixing syringe 500′ in FIG. 37. Mixing syringe 500′ comprises a syringe 430, a device 10′ to maintain a closed system while mixing, similar to mixing syringe with flush 500, a pressure actuated valved plunger 510′ and a plunger stem 546 and associated plunger 530′. Pressure actuated valved plunger 510′ is like valved plunger 510 in form and function except that valve stem 582′ of plunger 510′ does not comprise sensor tines 606 as no second valve opening is needed for mixing syringe 500′. However, mixing steps performed are identical with valved plunger 510′ actuation being identical to valved plunger 510 by application of pressure by force as indicated by arrows 658, 656 and 654 as seen in FIG. 25 and by displacement of diluent 574 from chamber 560 into chamber 550 to mix with a resident drug, such as drug 572. Note that associated plunger 530′ is structured to correspond to the interface provided by plate 584 of plunger 510′. Of course only drug should be dispensed from mixing syringe 500′. Also, for some mixing applications, safety precautions may not require properties of device 10′. In such applications, a conventional syringe which employs either valved plunger 510 or 510′ without a device 10′ may be made within the scope of the instant inventions disclosed herein.

### Material Considerations:

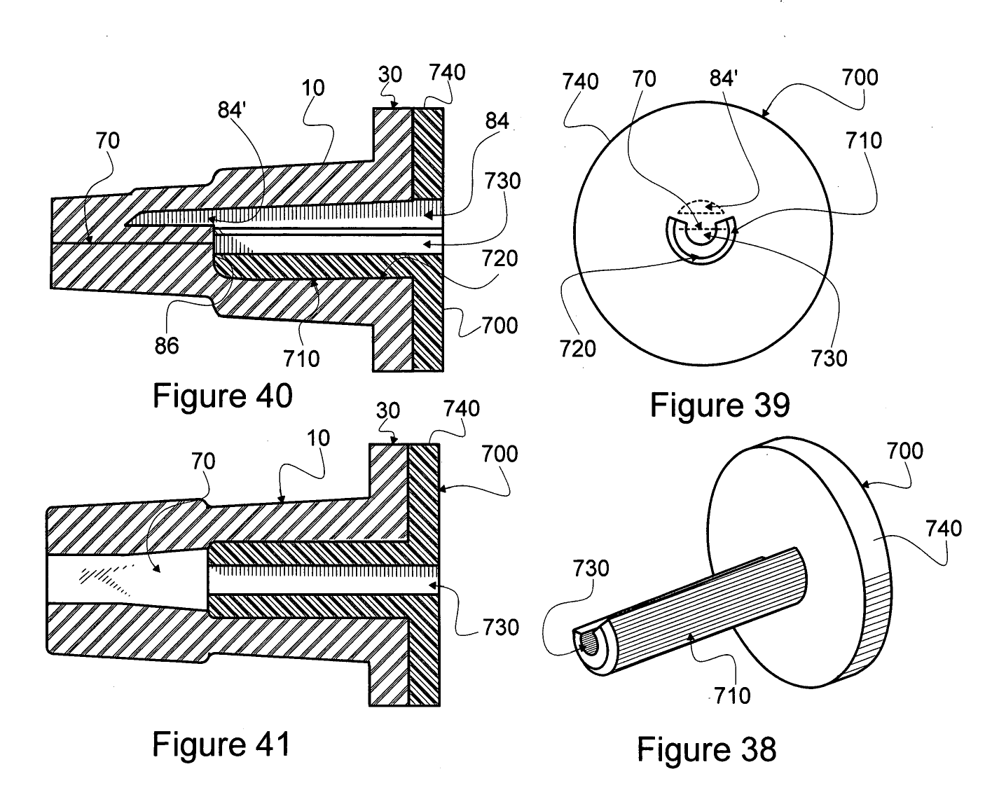

Material used in device 10 (see FIG. 1), or other like devices made according to the instant invention, should be comparatively stiff to assure device 10 fully inserts and does not pucker at areas where walls are thinned (see FIG. 2). In those cases where material is too supple to be effectively inserted, an internal support, such as support 700, seen in FIG. 38 may be used. Generally, support 700 may be injection molded using a rigid material such as polypropylene. A support member 710 which extends the full length of cavity 84 into contact with lip 86 (see FIGS. 2 and 40). Preferably, member 710 is cylindrical in shape with a superior portion removed to allow fluid communication with cavity 84′. Diameter of member 710 should provide a snug fit into the inferior portion 720 of cavity 84 while permitting fluid communication with cavity 84′. As seen in FIGS. 39 and 41, member 710 has a lengthwise trough 730 which is disposed to communicate with slit 70. Also, as seen in FIGS. 39 and 40, member 710 is not disposed to block cavity 84′. Member has a retention ring 740, as seen in FIGS. 38-41 which provides an anchor against attachment ring 30, as seen in FIG. 40.

### Molding and Slitting Considerations

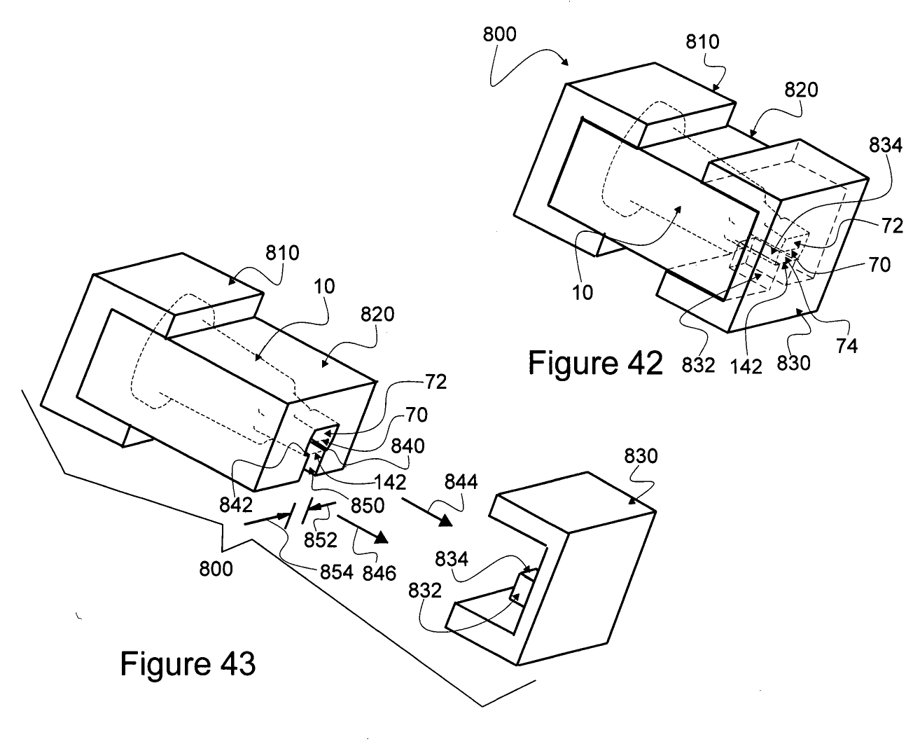

Thinness of a blade of slitting tool 360 (see FIG. 14) and suppleness of material of device 10 (or 10′) may result in producing a slit 70 having a pathway that wanders. Not only should stiffness of material of device 10 or 10′ be of careful consideration, but also retention of device geometry during slitting be a measure of concern to keep slit 70 as true as possible. For these reasons, molding devices 10 and 10′ should be guided by principles exhibited by an exemplary mold 800, seen in FIGS. 42 and 43, using device 10 as an example. Mold 800 is formed by three parts, aft portion 810, mid portion 820 and frontal portion 830. Aft portion provides for an inner mold part which defines cavities 84 and 84′ (not shown). Mid portion 820 defines the exterior of device 10, seen outlined by dashed lines, except for surface 142 (see also FIGS. 5 and 6) and face 122 (see also FIG. 1). Frontal portion 830 has an insertable tongue 832 having a superiorly disposed surface 834 which defines the major portion of surface 142 a flat vertical surface 836 which defines face 122. The rest of surface 142 is defined by two ledges 840 and 842, which are part of mid portion 820.

Before slitting, frontal portion 830 is displaced from mid portion 820 in direction of arrows 844 and 846 to open a rectangular slot 850, as seen in FIG. 43. For a 0.045 inch diameter hole width, per the example disclosed supra, width of slot 850, as defined by dimension arrows 852 and 854, should be on the order of 0.1 inch. Thus, after frontal portion is displaced, and slitting tool 360 is displaced to form slit 70, incompressible material in sections 40 and 50 is permitted to be relieved within slot 850, but rigorously held immobile otherwise especially by ledges 840 and 842, thereby constraining formation of slit 70 to as true a pathway as possible.

### CONCLUSION

Inventions disclosed herein may be embodied in other specific forms without departing from the spirit or essential characteristics thereof. The present embodiments are therefore to be considered in all respects as illustrative and not restrictive, the scope of the inventions being indicated by the appended claims rather than by the foregoing description, and all changes which come within the meaning and range of equivalency of the claims are therefore intended to be embraced therein.

What is claimed and desired to be secured by Letters Patent is:

1. A valve device which is self-closing and selectively opened for fluid passage, said valve device comprising:
a body comprising material having memory for returning from a distorted state to an originally molded form, said material further comprising characteristics of being incompressible, unreactive to contacting fluids, and resilient;
said body comprising a connecting end and an oppositely disposed valve end and an elongated medial axis between said ends, said valve end comprising two one-way valve parts oppositely disposed to provide a valve structure which is not opened by a fluid differential pressure applied about said axis;
said valve parts comprising a common slit there through, said slit being medially disposed within an outer surface comprising a cross-section of predetermined circumference;
said cross-section being asymmetric having an elongated length dimension in line with said slit and a width dimension orthogonally disposed relative to said slit;
said valve being closed when in a rest or undeformed state and opened, in one mode, by displacing said slit to an open state by deformation along the length dimension when inserted into a tapered fitting having circumferential dimensions which conform to said length and width dimensions.

2. A valving combination according to claim 2 comprising said valve device and a hollow cylindrical tapered tube having an end orifice sized to receive said valve and a cross-sectional circumference which deforms said elongated length dimension when said valve is disposed thereat to sufficiently part the slit and thereby open the valve.

3. A valving combination according to claim 2 wherein said circumference of said outer surface of said valve and said cross-sectional circumference of said tube correspond to provide a common mating interface.

4. A valving combination according to claim 2 wherein said hollow tapered tube is a female luer fitting.

5. A valving combination according to claim 2 wherein said circumference of said outer surface of said valve comprises dimensions of a male luer fitting when deformed within said female luer fitting.

6. A valve according to claim 1 wherein said material is butyl rubber.

7. A valve according to claim 1 wherein said slit is planar in form.

8. A method for making a tapered fitting valve which is self-closing and selectively opened for fluid passage comprising the steps of:
(a) providing a mold comprising an exterior mold part which comprises geometry which defines exterior shape and size of exterior surfaces of said valve, providing exterior surfaces of a most distal one-way valve and a more proximal one-way valve, each of said valves being disposed in a direction which opposes flow of the other valve, each of said valves being constructed to be opened by medially directed compression due to insertion into a tapered female fitting;
(b) providing an interior core mold part of the mold which comprises geometry which defines interior surfaces of said valve, said interior core mold part comprising an elongated channel through which a bladed part is displaced;
(c) providing a first elongated rod comprising a blunt end and comprising a shape and size for being displaced through said elongated channel;
(d) providing a second elongated rod comprising a sharpened end and comprising a shape and size for being displaced through said elongated channel, said second blade shaped part comprising a blade width which is consistent with a width requirement of a slit designed to be disposed within said one-way valves;
(e) displacing the first elongated rod into said channel to provide closure against channel filling during a molding process;
(f) molding said valve of synthetic resinous material which has memory for returning from a distorted state to an originally molded form, said material further comprising characteristics of being incompressible, unreactive to contacting fluids, pliant and resilient such that when said valve is inserted into a tapered female fitting, said valve is compressively distortable to fit snugly within the female fitting and when said valve is removed therefrom valve closure is reestablished;
(f) removing a portion of exterior molded part such that a slitting blade can be displaced through distally molded parts;
(g) removing the first elongated rod and displacing the second elongated rod through said channel and through said valve to form a common slit through each of said opposing one-way valves.

9. A method for making a tapered fitting valve according to claim 8 and for using the valve comprising the following steps:
adding an attachment to said valve mold whereby said molded valve comprises an interfacing collar;
providing a housing comprising components which provide a compressive seal about said collar, an associated female luer fitting and a fluid communicating pathway there between, thereby providing a stand-alone male luer fitting device.

10. A method for making a tapered fitting valve according to claim 8 and for using the valve comprising the following steps:
adding an appendage to said tapered fitting valve whereby said molded valve comprises an interface for a medical syringe whereby said valve can replace a male luer fitting of the medical syringe and thereby provide for fluid communication with said medical syringe only when said valve is inserted into a female luer fitting.

11. A method for making a closed system for mixing drugs according to claim 10 comprising the steps of:
providing:
(a) the medical syringe which is like a conventional medical syringe except for an open collar, sized and shaped for attachment of a tapered fitting valve to be disposed in place of a male luer fitting of a conventional syringe, said medical syringe comprising a barrel which is diminished in size at a distal end whereat said tapered fitting valve is disposed and open at a proximal end where a plunger stem and associated plunger combine to provide a fluid containment chamber within said barrel;
(b) the tapered fitting valve which provides a normally closed valve when unattached and opened for fluid flow by insertion into a female luer fitting; and
(c) a pressure actuated valved plunger, disposed within said barrel between said male luer adapter and said associated plunger to divide the fluid containment chamber into a distal chamber and a proximal chamber, which is actuated from a normally closed state by a pressure within the barrel which is greater than a differential pressure which provides sufficient force to displace said pressure actuated valved plunger along said barrel to a state which is open to flow in only a single direction from the proximal chamber to the distal chamber whereby fluid is displaced from the proximal chamber to mix with contents of the distal chamber.

12. A method for making a closed system for mixing and dispensing drugs with an associated flush according to claim 10 comprising the steps of:
providing:
(a) the medical syringe which is like a conventional medical syringe except for an open collar, sized and shaped for attachment of a tapered fitting valve to be disposed in place of a male luer fitting of a conventional syringe, said medical syringe comprising a barrel which is diminished in size at a distal end whereat said tapered fitting valve is disposed and open at a proximal end where a plunger stem and associated plunger combine to provide a fluid containment chamber within said barrel;
(b) the tapered fitting valve which provides a normally closed valve when unattached and opened for fluid flow by insertion into a female luer fitting;
(c) a pressure actuated valved plunger, disposed within said barrel between said male luer adapter and said associated plunger to divide the fluid containment chamber into a distal chamber and a proximal chamber, which is actuated from a normally closed state by a pressure within the barrel which is greater than a differential pressure which provides sufficient force to displace said pressure actuated valved plunger along said barrel to a state which is open to flow in only a single direction from the proximal chamber to the distal chamber whereby fluid is displaced from the proximal chamber to mix with contents of the distal chamber; and
(d) a valve stem disposed within said pressure actuated valved plunger which is displaced distally to a first state to provide a first open state which is the state which the pressure actuated valve is open in to flow in only a single direction and which is displaced proximally to a second state at which said valve is open to bidirectional flow; and
(e) a second valved plunger which is disposed within said barrel to divide the proximal chamber into a medial chamber and a most proximal chamber, the second valved plunger comprising a stem which is displaced proximally by proximal displacement of the valve stem of the pressure actuated valved plunger to provide a channel for displacing fluid from the most proximal chamber.

13. A closed mixing syringe system in which both drug and drug diluent is stored, transported, mixed and dispensed comprising:
(a) a medical syringe which is like a conventional medical syringe except for an open collar, sized and shaped for attachment of a tapered fitting valve to be disposed in place of a male luer fitting of a conventional syringe, said medical syringe comprising a barrel which is diminished in size at a distal end whereat said tapered fitting valve is disposed and open at a proximal end where a plunger stem and associated plunger combine to provide a fluid containment chamber within said barrel;
(b) the tapered fitting valve which provides a normally closed valve when unattached and opened for fluid flow by insertion into a female luer fitting;
(c) a pressure actuated valved plunger, disposed within said barrel between said male luer adapter and said associated plunger to divide the fluid containment chamber into a distal chamber and a proximal chamber, which is actuated from a normally closed state by a pressure within the barrel which is greater than a differential pressure which provides sufficient force to displace said pressure actuated valved plunger along said barrel to a state which is open to flow in only a single direction from the proximal chamber to the distal chamber whereby fluid is displaced from the proximal chamber to mix with contents of the distal chamber.

14. The closed mixing syringe system according to claim 13 wherein said pressure actuated valved plunger comprises a valve stem disposed within said pressure actuated valved plunger which is displaced distally to a first state to provide a first open state which is the state which the pressure actuated valve is open in to flow in only a single direction and which is displaced proximally to a second state at which said valve is open to bidirectional flow.

15. The closed mixing and flushing syringe system according to claim 14 wherein said system further comprises a second valved plunger which is disposed within said barrel to divide the proximal chamber into a medial chamber and a most proximal chamber, the second valved plunger comprising a stem which is displaced proximally by proximal displacement of the valve stem of the pressure actuated valved plunger to provide a channel for displacing fluid from the most proximal chamber.
